(19) United States
(12) Patent Application Publication
(10) Pub. No.: US 2022/0186268 A1
(43) Pub. Date: Jun. 16, 2022

(54) METHOD FOR PRODUCING CARBON-BASED PRODUCTS FROM SECONDARY RAW MATERIALS CONTAINING PH REGULATORS

(71) Applicant: BLUCON BIOTECH GMBH, Cologne (DE)

(72) Inventors: Vitaly Svetlichny, Cologne (DE); Marco Krämer, Pullheim (DE)

(21) Appl. No.: 17/440,064

(22) PCT Filed: Mar. 9, 2020
(86) PCT No.: PCT/EP2020/056197

§ 371 Date: Oct. 22, 2021

(30) Foreign Application Priority Data
Mar. 18, 2019 (DE) 10 2019 106 761.8

(51) Int. Cl. C12P 7/56; C12P 39/00; C12P 7/06; C12P 7/10

(52) U.S. Cl. CPC C12P 7/56; C12P 39/00; C12P 2203/00; C12P 7/10; C12P 7/065

(57) ABSTRACT

A process for the fermentative conversion of at least one cellulose and/or hemicellulose-containing secondary raw material into a carbon, in particular lactic acid-based product, the secondary raw material containing at least one pH regulator.

The present invention relates to methods for fermentatively converting at least one secondary raw material containing cellulose and/or hemicellulose into a carbon-based product, the secondary raw material containing at least one pH regulator.

### BACKGROUND OF THE INVENTION

The cultivated organisms used for the fermentative production of substances usually have a limited pH tolerance range that has an optimum pH. Pumps coupled to a pH sensor are usually used to control the pH value by means of pH regulators, which pumps pump acids such as phosphoric acid (H₃PO₄), hydrochloric acid (HCl) and others into the bioreactor in order to reduce the pH value or pump lyes such as caustic soda lye (NaOH), calcium hydroxide (Ca(OH)₂) and others into the bioreactor in order to increase the pH value, when necessary.

In addition, the pH value of a solution can be kept constant within a range by substances that have high acid binding capacity, meaning capacity to bind hydrogen ions. The substance calcium carbonate (CaCO₃) can be mentioned here as an example and is often used in biotechnological applications, for example in the fermentative production of lactic acid.

These pH regulators are therefore necessary for allowing for the optimum fermentative production of substances.

However, pH regulators generate production, purchase, transport and storage costs when fermentatively producing substances. These costs are associated with strains on the environment. Therefore, the transport of the pH regulators by means of internal combustion engines produces additional amounts of carbon dioxide, for example. Plot areas are required for the necessary storage of pH regulators, which increases sealing of the soil.

Among other things, the object of the present invention is to provide methods that make it possible to reduce the regulator.

### BRIEF DESCRIPTION OF THE INVENTION

The present invention relates to methods for fermentatively converting at least one secondary raw material, which is not pretreated using enzymes and contains cellulose and/or hemicellulose, into a carbon-based product, wherein the secondary raw material contains at least one pH regulator, said method comprising the step of bringing the secondary raw material into contact with a microorganism for a time period and at a starting temperature and an initial pH value, thereby producing an amount of lactic acid and/or a different carbon-based product.

In particular, the present invention describes the use of material flows that already exist in fermentation methods, such as the substrate (usable carbon sources), as pH regulators as a whole or elements of pH regulators. In addition to being used as carbon sources for the fermentative production of substances (for example lactic acid), secondary raw materials can therefore also directly involve regulators in the fermentation production as components for adjusting the pH value. Therefore, the addition of the pH regulator, such as calcium hydroxide, in the method can be reduced or avoided entirely.

It was surprisingly possible to establish that, by using paper sludges as the substrate, for example, efficient production of carbon-based products, in particular lactic acid, is possible using microorganisms such as Caldicellulosiruptor and/or Thermoanaerobacter, wherein the pH regulator, the number of moles of which normally has to be equal to that of the lactic acid produced, can be used in a manner in which there are considerably fewer moles thereof than of lactic acid or said pH regulator can even be completely dispensed of.

For example, groups of microorganisms, such as the group of Thermoanaerobacterales (e.g. Caldicellulosiruptor spec.) and Clostridiales (e.g. Clostridium thermocellum) can use papermaking residues containing regulators, in particular deinking sludges, which contain cellulose and hemicellulose as polymers and substrates, to produce lactic acid from cellulose and/or hemicellulose.

Furthermore, a coculture consisting of two organisms from the group of Thermoanaerobacterales (e.g. Caldicellulosiruptor spec. and Thermoanaerobacter spec.) can turn regulator-containing papermaking residues, in particular deinking sludges that contain cellulose and hemicellulose as polymers and substrates, into lactic acid.

### DETAILED DESCRIPTION OF THE INVENTION

Methods/processes for fermentatively converting at least one secondary raw material, which is not pretreated using enzymes and contains cellulose and/or hemicellulose, into a carbon-based product are described, the secondary raw material containing at least one pH regulator, said method comprising the step of bringing the secondary raw material into contact with a microorganism for a time period, at a starting temperature and an initial pH value, thereby producing an amount of lactic acid and/or a different carbon-based product.

Substrates in fermentation methods can be organic pure substances, organic by-products and organic secondary raw materials.
In chemistry, a pure substance is characterized as a substance that is uniformly composed of just one chemical compound or one chemical element.
A by-product is traditionally anything that is additionally, and often also undesirably, produced during the production of a (main) product.
Secondary raw materials are raw materials that are obtained by reprocessing (recycling) material that has been disposed of. They are used as starting materials for new products and thereby differ from the primary raw material (obtained from nature). When using renewable raw materials as substrates, this primarily relates to paper (wastepaper) and wood (wood waste).

Targetedly mixing substrates such as pure substances or by-products, for example those from agriculture, with regulators in the fermentation method is less expedient, since this method requires complex pretreatment, such as mixing the substrate, and the method is therefore commercially unappealing. In addition, by watering down and diluting the substrate using the regulator, overall higher amounts of the mixture of substrate and regulator are required here.

Some secondary raw materials (for example deinking residues), which comprise the polymers hemicellulose and cellulose, which can be used as substrates, originate from paper recycling.

The present invention is therefore directed to methods for fermentatively converting at least one secondary raw material, which is not pretreated using enzymes and contains cellulose and/or hemicellulose, into a carbon-based product, wherein the secondary raw material contains at least one pH regulator, said method comprising the step of bringing the secondary raw material into contact with a microorganism for a time period, at a starting temperature and an initial pH value, thereby producing an amount of lactic acid and/or a different carbon-based product.

More particularly, the carbon-based products produced by the method provided here are carboxylic acids, preferably lactic acid, or a salt or ester thereof.

In particular, within the context of the present invention, lactic acid is understood to mean hydroxycarboxylic acids, which have both a carboxyl group and a hydroxyl group and are more particularly also referred to as 2-hydroxypropionic acid. Furthermore, the hydroxycarboxylic acids referred to as 2-hydroxypropanoic acids in accordance with the nomenclature recommendations by the IUPAC are also understood to mean lactic acid within the context of the present invention. Furthermore, the present method also comprises the production of the salts and esters of lactic acids (lactates).

In another embodiment of the present invention, the carbon-based product can be an alcohol, preferably ethanol.

Within the context of the present invention, secondary raw material is, for example, papermaking residue, in particular deinking sludge from paper recycling. Within the context of the present invention, secondary raw material is, for example, papermaking residue, in particular fiber waste, fiber sludge, filler sludge and coating sludge from mechanical separation.

Within the context of the present invention, secondary raw material is, for example, papermaking residue, in particular sludge from treating wastewater from paper production.

Within the context of the present invention, secondary raw material is, for example, wastepaper, in particular packaging paper.

Within the context of the present invention, secondary raw material is plastic materials such as biodegradable plastics from renewable raw materials, in particular cellulose-based plastics having a composite content.

The deinking residues, known as deinking sludges, consist of fillers (calcium carbonate, kaolin, silicates), pulp (cellulose, hemicellulose and additional polymers), extractives (fats, soluble printing inks and coating color components) and fines (insoluble printing inks and coating color components, adhesive components). When using these substances, heat treatment (waste incineration) plays a central role. Almost all paper industry residues occur with relatively low solids contents, but due to the high content of organic components still generally possess such a high calorific value that they burn without a supplementary fire, i.e. energy is obtained. Therefore, more than 55% of deinking residues are burned as refuse-derived fuels in the paper mill's own power plants or are burned externally to generate power. The incombustible components are left in the form of (possibly usable) ash, clinker and filter dust.

Some secondary raw materials, for example all deinking sludges from paper recycling or all fiber waste, fiber sludges, filler sludges and coating sludges from mechanical separation, therefore already contain the regulator calcium carbonate.

In addition to being used as sources of carbon for the fermentative production of substances (for example lactic acid), these secondary raw materials can therefore also directly involve regulators in the fermentation method as components for adjusting the pH value. Therefore, the addition of the pH regulator, such as calcium hydroxide, in the method can be reduced or avoided entirely. The production costs can therefore be reduced.

Several secondary raw materials from the paper production process, such as deinking sludges from paper recycling and fiber waste, fiber sludges, filler sludges and coating sludges from mechanical separation, are currently incinerated. By using these raw materials as pH regulators, they no longer have a thermal use but a material use. Therefore, one environmental problem as a result of the reduction in the input of carbon (as CO₂) into the atmosphere is reduced.

In a preferred embodiment of the present invention, other than the pH regulator already present in the secondary raw material, no additional pH regulator is added to the method or only an amount of pH regulator is added to said method that contains fewer moles than the lactic acid produced.

As already described previously, the pH regulator present in the secondary raw material is, for example, CaCO₃, which improves the process and the costs are reduced by the process.

Particularly preferable embodiments of the present invention relate to methods for fermentatively converting at least one secondary raw material, which is not pretreated using enzymes and contains cellulose and/or hemicellulose, into a carbon-based product, the secondary raw material containing at least one pH regulator.

In particularly preferable embodiments of the present invention, in the present method no activities, or a lower or equal amount of activities, of enzymes that degrade cellulose and/or hemicellulose are added to the method, such as in fermentative methods with simultaneous saccharification and fermentation (SSF).

In particularly preferred embodiments of the present invention, hydrolases such as proteases, peptidases, phytases, glycosidases; cellulases, hemicellulases or combinations thereof are added to the method.

In particularly preferred embodiments of the present invention, isomerases such as racemases, epimerases and mutases or combinations thereof are added to the method.

In particularly preferred embodiments of the present invention, lyases such as aldolases, fumarases or combinations thereof are added to the method.

In particularly preferred embodiments of the present invention, the secondary raw material containing cellulose and/or hemicellulose is furthermore not pretreated using enzymes that degrade cellulose and/or hemicellulose before the method. Until now, paper sludges have been pretreated in the prior art by cellulases, for example.

In particularly preferred embodiments of the present invention, the microorganisms used in the claimed method belong to the group of Thermoanaerobacterales, in particular to the Caldicellulosiruptor genus, such as microorganisms from Table 1, or to the Thermoanaerobacter genus, such as microorganisms from Table 2.

TABLE 1

| | | | DSMZ | |
|---|---|---|---|---|
| | | | deposition | Deposition |
| Genus | Species | Name | number | date |
| Caldicellulosiruptor | sp. | DIB004C | DSM 25177 | 09/15/2011 |
| Caldicellulosiruptor | sp. | DIB041C | DSM 25771 | 03/15/2012 |
| Caldicellulosiruptor | sp. | DIB087C | DSM 25772 | 03/15/2012 |
| Caldicellulosiruptor | sp. | DIB101C | DSM 25178 | 09/15/2011 |
| Caldicellulosiruptor | sp. | DIB103C | DSM 25773 | 03/15/2012 |
| Caldicellulosiruptor | sp. | DIB104C | DSM 25774 | 03/15/2012 |
| Caldicellulosiruptor | sp. | DIB107C | DSM 25775 | 03/15/2012 |
| Caldicellulosiruptor | sp. | BluConL60 | DSM 33252 | 08/29/2019 |

TABLE 2

| | | | DSMZ | |
|---|---|---|---|---|
| | | | deposition | Deposition |
| Genus | Species | Name | number | date |
| Thermoanaerobacter | sp. | DIB004G | DSM 25179 | 09/15/2011 |
| Thermoanaerobacter | sp. | DIB087G | DSM 25777 | 03/15/2012 |
| Thermoanaerobacter | sp. | DIB097X | DSM 25308 | 10/27/2011 |
| Thermoanaerobacter | sp. | DIB101G | DSM 25180 | 09/15/2011 |
| Thermoanaerobacter | sp. | DIB101X | DSM 25181 | 09/15/2011 |
| Thermoanaerobacter | sp. | DIB103X | DSM 25776 | 03/15/2012 |
| Thermoanaerobacter | sp. | DIB104X | DSM 25778 | 03/15/2012 |
| Thermoanaerobacter | sp. | DIB107X | DSM 25779 | 03/15/2012 |

The strains DIB004C, DIB041C, DIB087C, DIB101C, DIB103C, DIB104C, DIB107C, DIB004G, DIB087G, DIB097X, DIB101G, DIB101X, DIB103X, DIB104X and DIB107X listed in Tables 1 and 2 were deposited under the above-mentioned registered DSMZ—entry numbers according to the requirements of the Budapest Treaty in relation to the deposition data provided for the DSMZ—German Collection of Microorganisms and Cell Cultures GmbH, Inhoffenstr. 7B, 38124 Braunschweig, Germany. The strain Caldicellulosiruptor sp. BluConL60 was deposited on 29 Aug. 2019 under the accession number DSM 33252 according to the requirements of the Budapest Treaty of the German Collection of Microorganisms and Cell Cultures (DSMZ), Inhoffenstraße 7B, 38124 Braunschweig, (DE), by BluCon Biotech GmbH, Nattermannallee 1, 50829, Cologne (DE).

The present invention therefore also comprises methods in which the microorganism is selected from the group consisting of DIB004C, deposited as DSM 25177, D1B041C, deposited as DSM 25771, D1B087C, deposited as DSM 25772, DIB101C, deposited as DSM 25178, DIB103C, deposited as DSM 25773, DIB104C, deposited as DSM 25774, BluConL60, deposited as DSM 33252 and DIB107C, deposited as DSM 25775.

Furthermore, the present invention also comprises methods in which the microorganism is selected from the group consisting of DIB004G, deposited as DSM 25179, DIB101G, deposited as DSM 25180, DIB101X, deposited as DSM 25181, D1B097X, deposited as DSM 25308, D1B087G, deposited as DSM 25777, D1B103X, deposited as DSM 25776, D1B104X, deposited as DSM 25778 and D1B107X, deposited as DSM 25779.

Furthermore, the present invention also comprises methods in which the microorganism in a coculture containing at least two different microorganisms from the group of Thermoanaerobacterales, in particular the Caldicellulosiruptor genus, such as microorganisms in Table 1, or the Thermoanaerobacter genus, such as microorganisms from Table 2.

Embodiments of the present invention therefore also comprise methods in which the microorganism and another microorganism in the form of a coculture are brought into contact with the secondary raw material. In particular, the additional microorganism can be a strain from Table 1 or Table 2.

In specific embodiments of the present invention, the microorganisms, which are used in the methods of the present disclosure, most efficiently grow and produce the carbon-based product at a specific starting temperature. In particular embodiments, one advantage of the methods of the present disclosure is the fact that the temperature can be high, preferably higher than 60° C., preferably 70° C. and higher, until a maximum temperature of 90° C., preferably 80° C., is reached, preferably 75° C., since the microorganisms used are thermophilic. This leads to a lower risk of contamination and to shorter reaction times.

In specific embodiments, the disclosure relates to any of the above-mentioned methods, wherein the time period is from approximately 10 hours to approximately 300 hours. In specific embodiments, the disclosure relates to any of the above-mentioned methods, wherein the time period is from approximately 50 hours to approximately 200 hours. In specific embodiments, the disclosure relates to any of the above-mentioned methods, wherein the time frame is from approximately 80 hours to approximately 160 hours. In specific embodiments, the disclosure relates to one of the above-mentioned methods, wherein the time period is approximately 80 hours, approximately 85 hours, approximately 90 hours, approximately 95 hours, approximately 100 hours, approximately 105 hours, approximately 110 hours, approximately 115 hours, approximately 120 hours, approximately 125 hours, approximately 130 hours, approximately 135 hours, approximately 140 hours, approximately 145 hours, approximately 150 hours, approximately 155 hours or approximately 160 hours. In a particularly preferred embodiment, the time period is from 70 h to 120 h.

In specific embodiments, the disclosure relates to any of the above-mentioned methods, wherein the time period is approximately 120 hours. In specific embodiments, the disclosure relates to any of the above-mentioned methods, wherein the starting temperature is from approximately 45° C. to approximately 80° C. In specific embodiments, the invention relates to any of the above-mentioned methods, wherein the starting temperature is from approximately 65° C. to approximately 80° C. In specific embodiments, the disclosure relates to any of the above-mentioned methods, wherein the starting temperature is from approximately 70° C. to approximately 75° C. In specific embodiments, the disclosure relates to any of the above-mentioned methods, wherein the starting temperature is approximately 72° C.

In specific embodiments, the disclosure relates to any of the above-mentioned methods, wherein the initial pH value is between approximately 5 and approximately 9. In specific embodiments, the disclosure relates to any of the above-mentioned methods, wherein the initial pH value is between approximately 6 and approximately 8. In specific embodiments, the disclosure relates to any of the above-mentioned methods, wherein the initial pH value is approximately 5, approximately 5.5, approximately 6, approximately 6.5, approximately 7, approximately 7.5, approximately 8, B. is approximately 8.5 or approximately 9. In specific embodiments, the disclosure relates to any of the above-mentioned methods, wherein the initial pH is approximately 6, approximately 6.5, approximately 7, approximately 7.5 or approximately 8.

In a specific embodiment, the starting temperature is between 65° C. and 80° C., the time period is 120 hours or longer and the initial pH value is between 6 and 8.

The invention will be described in more detail in the following on the basis of one embodiment, without limiting the general concept of the invention.

### Embodiment 1

This embodiment of the fermentative production of lactic acid by Caldicellulosiruptor, spec. D1B104C showed that the microbial substrate utilization of deinking sludge flotate suspensions as an example of a secondary raw material from the paper industry, which raw material contains hemicellulose and cellulose and contains the regulator CaCO₃, led to a reduction in the (external) alkaline regulator added when compared with cellulose as the pure substance (Avicel) without the regulator CaCO₃.

This can be attributed to the fact that the regulator, in this case CaCO₃, was already present in the cellulose-containing deinking sludge flotate.

The regulator therefore does not have to be produced and transported or only a much smaller amount has to be produced and transported. As a result, the method is more environmentally friendly and less expensive, since the regulator either does not have to be added to the method or a much smaller amount thereof has to be added to said method.

a1) Specification of Deinking Sludge Flotate

Result of the analysis of deinking sludge flotate (dry substance 70.1%). According to Sluiter et al., Determination of Structural Carbohydrates and Lignin in Biomass. Laboratory Analytical Procedure (LAP). Issue Date: April 2008. Revision Date July 2011 (Version Jul. 8, 2011). Enzymatic assay of xylose and glucose after hydrolysis using D-Xylose Assay Kit (K-XYLOSE) and D-Glucose HK Assay Kit (K-GLUHK-220A) by Megazyme, Ireland.

| Xylan | Cellulose | Xylan and cellulose |
|---|---|---|
| in 1000 g of dry | in 1000 g of dry | in 1000 g of dry |
| substance | substance | substance |
| 12 g | 72 g | 84 g |

a2) Specification of Avicel PH-101 (Cellulose Pure Substance), 11365, Sigma-Aldrich, Batch Number BCBW4188.

Avicel PH-101 (cellulose pure substance) by Sigma-Aldrich, batch number BCBW4188 has a dry weight of 95.5% (see certificate of analysis (CoA) by Sigma-Aldrich).

b) Calculation of the Amount of CaCO₃ in the Deinking Sludge Flotate

The deinking sludge flotate contains 183.98 g of Ca/kg of dry weight (=18.39%). This is 4.6 mol of Ca/kg of dry weight (molecular weight of Calcium-40). If said deinking sludge flotate equimolarly contains 4.6 mol of CO₃ (molecular weight of Carbonate 60), this is 275.97 g of CO₃/kg of dry weight. Overall, 459.95 g of calcium carbonate are therefore contained per kg of dry weight. The value of 46 g of CaCO₃/100 g of dry weight in the deinking sludge flotate was used for the statements.

c) Production of Dry Deinking Sludge Flotate

Approximately 300 g of deinking sludge flotate comprising 70.07% dry weight were dried for 4 days at 70° C. The dried deinking sludge flotate was then ground for 10 seconds using a coffee grinder (Clatronic KSW3306).

d) Cultivations

d1) Cultivation Batches

All cultivations were carried out in triplicate in serum bottles each having a volume of 110 ml:
Cultivations in batches 1a-c: dry deinking sludge flotate (having internal CaCO₃ as the regulator) was used as the substrate.
Cultivations in batches 2a-c: cellulose was used as the pure substance, Avicel PH-101 was used as the substrate.
Cultivations in batches 3a-c: cellulose was used as the pure substance, Avicel PH-101 and CaCO₃ were used as the external regulator (added).

d2) Addition of Substrate and Regulator

The following were added to empty serum bottles having a volume of 110 ml:
Each of batches 1a-c: 1.5 g of dry deinking sludge flotate (with internal CaCO₃ as the regulator)
Each of batches 2a-c: 0.16 g of Avicel PH-101, 11365, Sigma-Aldrich, batch number BCBW4188).
Each of batches 3a-c: 0.16 g of Avicel PH-101, 11365, Sigma-Aldrich, batch number BCBW4188) and 0.7 g of CACO₃ (Roth, P013.2, batch number 137253672, used as the regulator.

d3) Production of the Resazurin Stock Solution:

Resazurin is an indicator, which is used for redox reactions. In the non-reduced state, the solution is blue; under anaerobic conditions and with the addition of L-cysteine, the solution turns colorless. Concentration/resazurin:

50 mg/50 ml VE-H₂O, storage at +4° C. Resazurin, Na salt, Acros 418900050

d4) Production of the Trace Element Parent Solution:

| | | Concentration | | Concentration |
|---|---|---|---|---|
| | | in the medium | | in the parent |
| | | [mg/l of | | solution |
| No. | Substance | medium] | Manufacturer | [g/l] |
| 1 | NiCl₂ × 6H₂O | 1 | Roth 4489.1 | 2.0 |
| 2 | FeSO₄ × 7H₂O | 0.5 | Sigma-Aldrich | 1.0 |
| | | | 12354 | |
| 3 | NH₄Fe(III) | 5 | Roth CN77.1 | 10.0 |
| | citrate, approx. | | | |
| | 18% Fe | | | |
| 4 | MnSO₄ × H₂O | 2.5 | Sigma-Aldrich | 5.0 |
| | | | 13245 | |
| 5 | CoCl₂ × 6H₂O | 0.5 | Roth 7095.1 | 1.0 |
| 6 | ZnSO₄ × 7H₂O | 0.5 | Sigma-Aldrich | 1.0 |
| | | | 14455 | |
| 7 | CuSO₄ × 5H₂O | 0.05 | Roth 8175.1 | 0.1 |
| 8 | H₃BO₃ | 0.05 | Roth P010.1 | 0.1 |
| 9 | Na₂MoO₄ × 2H₂O | 0.065 | Roth 0274.1 | 0.1 |
| 10 | Na₂SeO₃ × 5H₂O | 0.05 | Sigma-Aldrich | 0.2 |
| | | | S5261 | |
| 11 | Na₂WoO₄ × 2H₂O | 0.05 | Sigma-Aldrich | 0.1 |
| | | | 72069 | |
| 12 | Deionized water | | | to 1000 ml |

After addition of the salt components, the trace element solution has a pH value of approximately 4.8. In order to dissolve all the salts, HCl, 32% (Roth X896.1) was added in a volume of 1 ml/l of trace element solution, thus then decreasing the pH value to 3.2.

d5) Production of the Basic Medium

| | | | Concentration in |
|---|---|---|---|
| No. | Substance | Manufacturer | the medium [g/l] |
| 1 | NH₄Cl | Roth K298.3 | 2.0 |
| 2 | NaCl | Applichem 201659 | 0.25 |
| 3 | MgSO₄ × 7 H₂O | Roth P027.2 | 1.35 |
| 4 | CaCl₂ × 2 H₂O | Roth 5239.1 | 0.5 |
| 5 | NaHCO₃ | VWR 27.778.236 | 0.25 |
| 6 | K₂HPO₄ | VWR 26.931.263 | 0.75 |
| 7 | KH₂PO₄ | VWR 0781 | 1.5 |
| 8 | Yeast extract | BD Bacto 212750 | 0.5 |
| 9 | Meat extract | Sigma 70164 | 1.0 |
| 10 | Trace element | s.a. | 0.5 ml/l |
| 11 | Resazurin parent solution | s.a. | 0.25 mg/l |
| 12 | VE-H₂O | | to 1 I |

d6) Production of the Cultivation Media/Cultivation Batches
After the production of the basic medium (see above), the pH value was adjusted to 6.5 (at 23° C.) using 5 N NaOH.
It is gassed with N₂ for 20 minutes while stirring. After gassing, 0.5 g of L-cysteine are added per liter of the medium.
While gassing with N₂, meter 30 ml of the medium into serum bottles comprising substrate and regulator (see above) while supplying nitrogen. Close the serum bottles using black butyl rubber bungs and aluminum cap and autoclave for 20 minutes at 121° C. and under 1 bar of overpressure.

The cultivation batches therefore contain the following usable substrates as the polymers cellulose and xylan, each calculated as a glucose and xylose equivalent, and regulator:
Each of batches 1a-c: 47.6 g/l of dry deinking sludge flotate (contains 21.9 g/l of CaCO₃ as the regulator) with the substrates 19.4 mM of glucose equivalents, 3.8 mM of xylose equivalents, from which a maximum of 45.4 mM of products (such as lactic acid and others) could be produced.
Each of batches 2a-c: 5.08 g/l of Avicel without regulator, with the substrate 31.4 mM of glucose equivalents, from which a maximum of 62.7 mM of products (such as lactic acid and others) could be produced.
Each of batches 3a-c: 5.08 g/l of Avicel with 22.2 g/l of CaCO₃ regulator with the substrate 31.4 mM of glucose equivalents, from which a maximum of 62.7 mM of products (such as lactic acid and others) could be produced.

d7) Production of a Preculture

100 ml of basic medium for precultures were produced with 10 g/l of Avicel and 0.5 g/l of L-cysteine in 250-ml serum bottles, as shown above.

The preculture medium was inoculated with 8 ml of a Working Cell Bank (storage at −30° C.) of Caldicellulosiruptor spec., D1B104C and cultivated for 24 h at 70° C. and 130 rpm in a shaking incubator.

d8) Inoculation of the Cultivation Batches and Sampling

The cultivation batches 1a-c, 2a-c and 3a-c were inoculated with 1.5 ml of the preculture and incubated for 5 days at 70° C. without shaking.

d9) Sampling

2-ml samples were taken from the cultivation batches in a sterile manner, the pH value was determined using a pH meter (by inoLab) and said samples were then transferred to a micro-reaction vessel and centrifuged at 16,000 g. The supernatants were each removed using a pipette and transferred to a new micro-reaction vessel.

d10) Analyses of the Supernatants

The supernatants were diluted with equal volumes of 1.5 M HCl and each transferred to an HPLC Vial (1.5 ml KGW bottle, brown 1 VWR product no. 548-0030) having a lid (9 mm PP KGW cap red hole PTFE VIRG 53° VWR product no. 548-0839). 30 μl of the sample were injected into an HPLC system (Shimadzu LabSolutions; Software: LabSolutions; Pump: LC-20AD, Auto-Sampler: SIL-20AC; oven CTO-20A and RI Detector: RID-20A) with a Rezex ROA-Organic Acid H+ (8%) HPLC column by Phenomenex and using a precolumn Carbo-H4×3.0 mm AJ0-4490 and the SecurityGuard Guard Cartridge Kit KJ0-4282. The concentration of lactic acid was determined by means of a reference calibration series using sodium L-lactic acid (by Applichem A1004,0100) 60, 30, 15, 7.5 and 3.25 g/l of sodium L-lactic acid, which is 46.6; 23.3; 11.65; 5.83 and 2.913 g/l of lactic acid. The concentrations of lactic acid determined were converted from g/l into mM.

e) Results of the Samples after Cultivation for 5 Days

The pH values determined are shown in Table 3:

TABLE 3
Results of the determination of the pH values of the cultures after
cultivation for 5 days.

| Batch no. | Substrate and regulator | pH value |
|---|---|---|
| 1a | Deinking sludge flotate (contains regulator) | 6.09 |
| | without external regulator | |
| 1b | Deinking sludge flotate (contains regulator) | 6.09 |
| | without external regulator | |
| 1c | Deinking sludge flotate (contains regulator) | 6.13 |
| | without external regulator | |
| | Average pH of batches 1a to 1c | 6.10 |
| 2a | Avicel without external regulator | 4.75 |
| 2b | Avicel without external regulator | 4.74 |
| 2c | Avicel without external regulator | 4.72 |
| | Average pH of batches 2a to 2c | 4.74 |
| 3a | Avicel with external regulator, CaCO₃ | 6.49 |
| 3b | Avicel with external regulator, CaCO₃ | 6.57 |
| 3c | Avicel with external regulator, CaCO₃ | 6.48 |
| | Average pH of batches 3a to 3c | 6.51 |

The result showed that, without the addition of a regulator, the pH value sunk to below pH 5 (batches 2a-2c). This is the pH range within which Caldicellulosiruptor spec. DIB104C is no longer physiologically active.

In the presence of a regulator, which was either already present in the secondary raw material in the deinking sludge flotate (contains CaCO₃ as the regulator) or was externally added as CaCO₃, in contrast the pH value was held in the physiological range (pH between pH 6 and pH 8) for Caldicellulosiruptor spec. DIB104C (batches 1a-1c and 3a-3c).

The addition of a regulator, either externally as CaCO₃ or as a component of the hemicellulose- and cellulose-containing secondary raw material from the paper industry, was therefore necessary to set the physiological range for Caldicellulosiruptor, spec. DIB104C (pH between pH 6 and pH 8).

The specific lactic acid concentrations are shown in Table 4:

TABLE 4
Results of the determination of lactic acid in cell-free supernatants of
the cultures after cultivation for 5 days.

| Batch | | Lactic acid |
|---|---|---|
| no. | Substrate and regulator | [mM] |
| 1a | Deinking sludge flotate (contains regulator) | 12.97 |
| | without external regulator | |
| 1b | Deinking sludge flotate (contains regulator) | 12.57 |
| | without external regulator | |
| 1c | Deinking sludge flotate (contains regulator) | 12.21 |
| | without external regulator | |
| | Average lactic acid concentration of batches 1a to 1c | 12.58 |
| 2a | Avicel without external regulator | 5.77 |
| 2b | Avicel without external regulator | 5.79 |
| 2c | Avicel without external regulator | 5.55 |
| | Average lactic acid concentration of batches 2a to 2c | 5.70 |
| 3a | Avicel with external regulator, CaCO₃ | >12 |
| 3b | Avicel with external regulator, CaCO₃ | >12 |
| 3c | Avicel with external regulator, CaCO₃ | >12 |
| | Average lactic acid concentration of batches 3a to 3c | >12 |

The result showed that, without the addition of a regulator, the lactic acid concentration was on average 5.70 mM (batches 2a-2c).

In the presence of a regulator, which was either already present in the secondary raw material in the deinking sludge flotate (contains CaCO₃ as the regulator) or was externally added as CaCO₃, in contrast an average lactic acid concentration of 12.58 mM was reached in the deinking sludge flotate (batches 1a-1c) and, using CaCO₃ (externally added), a lactic acid concentration higher than 12 mM was reached. This is more than double the concentrations reached without a regulator.

The addition of a regulator therefore consequently led to the adjustment of the pH value by means of the regulator to within the physiological pH range for Caldicellulosiruptor spec. DIB104C and to an increase in the lactic acid concentration. The addition of the regulator is therefore necessary for the efficient production of lactic acid.

Both the addition of the regulator external to the substrate Avicel and the use of a substrate, deinking sludge flotate, that already contains the regulator, led to an increase in the lactic acid concentration. Therefore, in the present example, it was advantageous to use the substrate deinking sludge flotate, which already contains the regulator, since this led to a reduction in the externally added regulator, CaCO₃.

The externally added regulator thus did not have to be produced and transported, or only a much smaller amount thereof had to be produced and transported. As a result, the method is more environmentally friendly and less expensive, since the regulator either did not have to be supplied to the method or only a much smaller amount thereof had to be supplied to said method.

### Embodiment 2

In embodiment 2, the microorganism Caldicellulosiruptor sp. strain BluConL60, was used, which was deposited on 29 Aug. 2019 by BluCon Biotech GmbH, Nattermannallee 1, 50829, Cologne (DE) under the accession number DSM 33252 according to the requirements of the Budapest Treaty of the German Collection of Microorganisms and Cell Cultures (DSZM), Inhoffenstraße 7B, 38124 Braunschweig (DE).

This embodiment of the fermentative production of lactic acid by Caldicellulosiruptor, spec. strain BluConL60 showed that the microbial substrate utilization of deinking sludge flotate suspensions as an example of a secondary raw material from the paper industry, which raw material contains hemicellulose and cellulose and contains the regulator CaCO₃, led to a reduction in the (external) alkaline regulator added when compared with cellulose as the pure substance (Avicel) without the regulator CaCO₃.

This can be attributed to the fact that the regulator, in this case CaCO₃, was already present in the cellulose-containing deinking sludge flotate. The regulator therefore does not have to be produced and transported or only a much smaller amount has to be produced and transported. As a result, the method is more environmentally friendly and less expensive, since the regulator either does not have to be added to the method or a much smaller amount thereof has to be added to said method.

a1) Specification of Deinking Sludge Flotate

Result of the analysis of deinking sludge flotate (dry substance 70.1%). According to Sluiter et al., Determination of Structural Carbohydrates and Lignin in Biomass. Laboratory Analytical Procedure (LAP). Issue Date: April 2008. Revision Date July 2011 (Version Jul. 8, 2011). Enzymatic assay of xylose and glucose after hydrolysis using D-Xylose Assay Kit (K-XYLOSE) and D-Glucose HK Assay Kit (K-GLUHK-220A) by Megazyme, Ireland.

| Xylan | Cellulose | Xylan and cellulose |
|---|---|---|
| in 1000 g of dry | in 1000 g of dry | in 1000 g of dry |
| substance | substance | substance |
| 12 g | 72 g | 84 g |

a2) Specification of Avicel PH-101 (Cellulose Pure Substance), 11365, Sigma-Aldrich, Batch Number BCCB8451.

Avicel PH-101 (cellulose pure substance) by Sigma-Aldrich, (product number 11365), batch number BCCB8451, has a dry weight of 96% (see certificate of analysis (CoA) by Sigma-Aldrich).

b) Calculation of the Amount of CaCO₃ in the Deinking Sludge Flotate

The deinking sludge flotate contains 183.98 g of Ca/kg of dry weight (=18.39%). This is 4.6 mol of Ca/kg of dry weight (molecular weight of Calcium 40). If said deinking sludge flotate equimolarly contains 4.6 mol of CO₃ (molecular weight of Carbonate 60), this is 275.97 g of CO₃/kg of dry weight. Overall, 459.95 g of calcium carbonate are therefore contained per kg of dry weight. The value of 46 g of CaCO₃/100 g of dry weight in the deinking sludge flotate was used for the statements.

c) Production of Dry Deinking Sludge Flotate

Approximately 300 g of deinking sludge flotate comprising 70.07% dry weight were dried for 4 days at 70° C. The dried deinking sludge flotate was then ground for 10 seconds using a coffee grinder (Clatronic KSW3306).

d) Cultivations

d1) Cultivation Batches

All cultivations were carried out in triplicate in serum bottles each having a volume of 110 ml:
Cultivations in batches 1a-c: dry deinking sludge flotate (having internal CaCO₃ as the regulator) was used as the substrate.
Cultivations in batches 2a-c: cellulose was used as the pure substance, Avicel PH-101 was used as the substrate.
Cultivations in batches 3a-c: cellulose was used as the pure substance, Avicel PH-101 and CaCO₃ were used as the external regulator (added).

d2) Addition of Substrate and Regulator

The following were added to empty serum bottles having a volume of 110 ml:
Each of batches 1a-c: 1.5 g of dry deinking sludge flotate (with internal CaCO₃ as the regulator)
Each of batches 2a-c: 0.16 g of Avicel PH-101, 11365, Sigma-Aldrich, batch number BCCB8451).
Each of batches 3a-c: 0.16 g of Avicel PH-101, 11365, Sigma-Aldrich, batch number BCCB8451) and 0.7 g of CaCO3 (Acros Organics, 450680010), used as the regulator.
Each of the bottles containing batches 1a-c, 2a-c and 3a-c were gassed for approximately 20 seconds while adding nitrogen, subsequently closed using a butyl rubber bung and then incubated for 1 to 2 hours at room temperature.

d3) Production of the Resazurin Stock Solution:

Resazurin is an indicator, which is used for redox reactions. In the non-reduced state, the solution is blue; under anaerobic conditions and with the addition of L-cysteine (by Roth 1693.3), the solution turns colorless. Concentration/resazurin:

50 mg/50 ml VE-H₂O, storage at +4° C. Resazurin, Na salt, Acros Organics 418900050

d4) Production of the Trace Element Parent Solution:

| | | Concentration | | Concentration |
|---|---|---|---|---|
| | | in the medium | | in the parent |
| No. | Substance | [mg/l of medium] | Manufacturer | solution [g/l] |
| 1 | NiCl₂ × 6H₂O | 1 | Roth 4489.1 | 2.0 |
| 2 | FeSO₄ × 7H₂O | 0.5 | Sigma-Aldrich | 1.0 |
| | | | 12354 | |
| 3 | NH₄Fe(III) | 5 | Roth CN77.1 | 10.0 |
| | citrate, approx. | | | |
| | 18% Fe | | | |
| 4 | MnSO₄ × H₂O | 2.5 | Sigma-Aldrich | 5.0 |
| | | | 13245 | |
| 5 | CoCl₂ × 6H₂O | 0.5 | Roth 7095.1 | 1.0 |
| 6 | ZnSO₄ × 7H₂O | 0.5 | Sigma-Aldrich | 1.0 |
| | | | 14455 | |
| 7 | CuSO₄ × 5H₂O | 0.05 | Roth 8175.1 | 0.1 |
| 8 | H₃BO₃ | 0.05 | Roth P010.1 | 0.1 |
| 9 | Na₂MoO₄ × 2H₂O | 0.065 | Roth 0274.1 | 0.1 |
| 10 | Na₂SeO₃ × 5H₂O | 0.05 | Sigma-Aldrich | 0.2 |
| | | | S5261 | |
| 11 | Na₂WoO₄ × 2H₂O | 0.05 | Sigma-Aldrich | 0.1 |
| | | | 72069 | |
| 12 | Deionized water | | | to 1000 ml |

After addition of the salt components, the trace element solution has a pH value of approximately 4.8. In order to dissolve all the salts, HCl, 32% (Roth X896.1) was added in a volume of 1 ml/l of trace element solution, thus then decreasing the pH value to 3.2.

d5) Production of the Vitamin Parent Solution:

| | | Concentration | | Concentration |
|---|---|---|---|---|
| | | in the medium | | in the parent |
| No. | Substance | [mg/l of medium] | Manufacturer | solution [g/l] |
| 1 | Nicotinic acid | 1 | Acros | 1 |
| | | | Organics | |
| | | | 380325000 | |
| 2 | Cyanocobalamin | 0.125 | Acros | 0.125 |
| | (B12) | | Organics | |
| | | | 405920010 | |
| 3 | p-aminobenzoic | 0.125 | Acros | 0.125 |
| | acid (4- | | Organics | |
| | aminobenzoic | | 146212500 | |
| | acid) | | | |
| 4 | Calcium D- | 0.125 | Acros | 0.125 |
| | pantothenate | | Organics | |
| | | | 243301000 | |
| 5 | Thiamine HCI | 0.125 | Acros | 0.125 |
| | | | Organics | |
| | | | 148990100 | |
| 6 | Riboflavin (B2) | 0.125 | Acros | 0.125 |
| | | | Organics | |
| | | | 132351000 | |
| 7 | Lipoic acid | 0.125 | Acros | 0.125 |
| | | | Organics | |
| | | | 138720050 | |
| 8 | Folic acid | 0.05 | Acros | 0.05 |
| | | | Organics | |
| | | | 216630100 | |
| 9 | Biotin (vitamin H) | 0.05 | Acros | 0.05 |
| | | | Organics | |
| | | | 230095000 | |
| 10 | Pyridoxine HCl | 0.05 | Acros | 0.05 |
| | (B6) | | Organics | |
| | | | 150770500 | |
| 11 | Deionized water | | | to 1000 ml |

All components are mixed in 1 liter of deionized water; the vitamin parent solution is cloudy due to riboflavin. The solution is filtered in a sterile manner using a filter having a pore size of 0.2 urn. The parent solution is then transparent. The vitamin parent solution is stored at +4° C.

D6) Production of the Basic Medium

| | | | Concentration in the |
|---|---|---|---|
| No. | Substance | Manufacturer | medium [g/l] |
| 1 | NH₄Cl | Roth K298.3 | 2.0 |
| 2 | NaCl | Applichem 201659 | 0.25 |
| 3 | MgSO₄ × 7 H₂O | Roth P027.2 | 1.35 |
| 4 | CaCl₂ × 2 H₂O | Roth 5239.1 | 0.5 |
| 5 | NaHCO₃ | VWR 27.778.236 | 0.25 |
| 6 | K₂HPO₄ | VWR 26.931.263 | 0.75 |
| 7 | KH₂PO₄ | VWR 0781 | 1.5 |
| 8 | Yeast extract | BD Bacto 212750 | 0.5 |
| 9 | Meat extract | Sigma 70164 | 1.0 |
| 10 | Trace element | s.a. | 0.5 ml/l |
| | parent solution | | |
| 11 | Vitamin parent | s.a. | 1 ml/l |
| | solution | | |
| 12 | Resazurin parent | s.a. | 0.25 mg/l |
| | solution | | |
| 13 | VE-H₂O | | to 1 I |

d7) Production of the Cultivation Media/Cultivation Batches
After the production, the basic medium (see above) had a pH value of 6.38.
It is gassed with N₂ for 20 minutes while stirring. After gassing, 0.5 g of L-cysteine are added per liter of the medium.
After the addition of L-cysteine, the medium has a pH value of 6.53.
While gassing with N₂, meter 30 ml of the medium into serum bottles comprising substrate and regulator (see above) while supplying nitrogen. Close the serum bottles using black butyl rubber bungs and aluminum cap and autoclave for 20 minutes at 121° C. and under 1 bar of overpressure.

The cultivation batches therefore contain the following usable substrates as the polymers cellulose and xylan, each calculated as a glucose and xylose equivalent, and regulator:
Each of batches 1a-c: 47.6 g/l of dry deinking sludge flotate (contains 21.9 g/l of CaCO₃ as the regulator) with the substrates 19.4 mM of glucose equivalents, 3.8 mM of xylose equivalents, from which a maximum of 45.4 mM of products (such as lactic acid and others) could be produced.
Each of batches 2a-c: 5.08 g/l of Avicel without regulator, with the substrate 31.4 mM of glucose equivalents, from which a maximum of 62.7 mM of products (such as lactic acid and others) could be produced.
Each of batches 3a-c: 5.08 g/l of Avicel with 22.2 g/l of CaCO₃ regulator with the substrate 31.4 mM of glucose equivalents, from which a maximum of 62.7 mM of products (such as lactic acid and others) could be produced.

d8) Production of a Preculture

100 ml of basic medium for precultures were produced with 10 g/l of Avicel and 0.5 g/l of L-cysteine in 250-ml serum bottles, as shown above.

The preculture medium was inoculated with 8 ml of a Working Cell Bank (storage at −30° C.) of Caldicellulosiruptor spec., strain BluConL60, and cultivated for 24 h at 70° C. and 130 rpm in a shaking incubator.

d9) Inoculation of the Cultivation Batches and Sampling

The cultivation batches 1a-c, 2a-c and 3a-c were inoculated with 1.5 ml of the preculture and incubated for 11 days at 70° C. without shaking.

d10) Sampling

2-ml samples were taken from the cultivation batches after 5 days and after 11 days in a sterile manner, the pH value was determined using a pH meter (by inoLab) and the samples were then transferred to a micro-reaction vessel and centrifuged at 16,000 g. The supernatants were each removed using a pipette and transferred to a new micro-reaction vessel.

d11) Analyses of the Supernatants

The supernatants were diluted with equal volumes of 2.5 mM H₂SO₄ and each transferred to an HPLC Vial (1.5 ml KGW bottle, brown 1 VWR product no. 548-0030) having a lid (9 mm PP KGW cap red hole PTFE VIRG 53° VWR product no. 548-0839). 30 μl of the sample were injected into an HPLC system (Shimadzu LabSolutions; Software: LabSolutions; Pump: LC-20AD, Auto-Sampler: SIL-20AC; oven CTO-20A and RI Detector: RID-20A) with a Rezex ROA-Organic Acid H+(8%) HPLC column by Phenomenex and using a precolumn Carbo-H4×3.0 mm AJ0-4490 and the SecurityGuard Guard Cartridge Kit KJ0-4282. The concentration of lactic acid was determined by means of a reference calibration series using sodium L-lactic acid (by Applichem A1004,0100) 60, 30, 15, 7.5 and 3.25 g/l of sodium L-lactic acid, which is 46.6; 23.3; 11.65; 5.83 and 2.913 g/l of lactic acid. The concentrations of lactic acid determined were converted from g/I into mM.

e) Results of the Samples after Cultivation for 5 Days and 11 Days

The pH values determined are shown in Table 5:

TABLE 5
Results of the determination of the pH values of the cultures after
cultivation for 5 days and 11 days.

| | | pH value | pH value |
|---|---|---|---|
| Batch | | after 5 | after 11 |
| no. | Substrate and regulator | days | days |
| 1a | Deinking sludge flotate (contains regulator) | 6.32 | 6.02 |
| | without external regulator | | |
| 1b | Deinking sludge flotate (contains regulator) | 6.29 | 6.01 |
| | without external regulator | | |
| 1c | Deinking sludge flotate (contains regulator) | 6.30 | 5.97 |
| | without external regulator | | |
| | Average pH of batches 1a to 1c | 6.30 | 6.00 |
| 2a | Avicel without external regulator | 4.89 | 5.09 |
| 2b | Avicel without external regulator | 4.89 | 4.80 |
| 2c | Avicel without external regulator | 4.86 | 4.79 |
| | Average pH of batches 2a to 2c | 4.88 | 4.89 |
| 3a | Avicel with external regulator, CaCO₃ | 6.35 | 6.33 |
| 3b | Avicel with external regulator,CaCO₃ | 6.35 | 6.29 |
| 3c | Avicel with external regulator, CaCO₃ | 6.35 | 6.34 |
| | Average pH of batches 3a to 3c | 6.35 | 6.32 |

The result showed that, without the addition of a regulator, the pH value sunk to below pH 5.1 (batches 2a-2c). This is the pH range within which Caldicellulosiruptor, spec. strain BlueConL60, is no longer physiologically active.

In the presence of a regulator, which was either already present in the secondary raw material in the deinking sludge flotate (contains CaCO₃ as the regulator) or was externally added as CaCO₃, in contrast the pH value was held in the physiological range (pH between pH 6 and pH 8) for Caldicellulosiruptor, spec. strain BlueConL60 (batches 1a-1c and 3a-3c).

The addition of a regulator, either externally as CaCO₃ or as a component of the hemicellulose- and cellulose-containing secondary raw material from the paper industry, was therefore necessary to set the physiological range for Caldicellulosiruptor, spec. strain BlueConL60 (pH between pH 6 and pH 8).

The specific lactic acid concentrations are shown in Table 6:

TABLE 6
Results of the determination of lactic acid in cell-free supernatants
of the cultures after cultivation for 5 days and 11 days.

| | | Lactic | Lactic |
|---|---|---|---|
| | | acid | acid |
| | | [mM] | [mM] |
| Batch | | after | after |
| no. | Substrate and regulator | 5 days | 11 days |
| 1a | Deinking sludge flotate (contains regulator) | 10.90 | 21.63 |
| | without external regulator | | |
| 1b | Deinking sludge flotate (contains regulator) | 8.50 | 19.40 |
| | without external regulator | | |
| 1c | Deinking sludge flotate (contains regulator) | 9.81 | 19.52 |
| | without external regulator | | |
| | Average lactic acid concentration of batches | 9.73 | 20.18 |
| | 1a to 1c | | |
| 2a | Avicel without external regulator | 6.62 | 6.90 |
| 2b | Avicel without external regulator | 7.15 | 7.26 |
| 2c | Avicel without external regulator | 7.24 | 7.93 |
| | Average lactic acid concentration of batches | 7.00 | 7.37 |
| | 2a to 2c | | |
| 3a | Avicel with external regulator, CaCO₃ | >20 | >20 |
| 3b | Avicel with external regulator, CaCO₃ | >20 | >20 |
| 3c | Avicel with external regulator, CaCO₃ | >20 | >20 |
| | Average lactic acid concentration of batches | >20 | >20 |
| | 3a to 3c | | |

The result showed that, without the addition of a regulator, the average lactic acid concentration was 7.00 mM after 5 days and 7.37 mM after 11 days (batches 2a-2c).

In the presence of a regulator, which was either already present in the secondary raw material in the deinking sludge flotate (contains CaCO₃ as the regulator) or was externally added as CaCO₃, in contrast an average lactic acid concentration of 9.73 mM after 5 days and 20.18 mM after 11 days was reached in the deinking sludge flotate (batches 1a-1c) and, using CaCO₃ (externally added), a lactic acid concentration higher than 20 mM was reached after 5 days and after 11 days (batches 3a to 3c). This is more than double the concentrations reached without a regulator.

The addition of a regulator therefore consequently led to the pH value being set within the physiological pH range for Caldicellulosiruptor, spec. strain BluConL60 by means of the regulator, and to the lactic acid concentration being increased. The addition of the regulator is therefore necessary for the efficient production of lactic acid.

Both the addition of the regulator external to the substrate Avicel and the use of a substrate, deinking sludge flotate, that already contains the regulator, led to an increase in the lactic acid concentration.

Therefore, in the present example, it was advantageous to use the substrate deinking sludge flotate, which already contains the regulator, since this led to a reduction in the externally added regulator, CaCO₃.

The externally added regulator thus did not have to be produced and transported, or only a much smaller amount thereof had to be produced and transported. As a result, the method is more environmentally friendly and less expensive, since the regulator either did not have to be supplied to the method or only a much smaller amount thereof had to be supplied to said method.

1. A method for fermentatively converting at least one secondary raw material, which is not pretreated using enzymes and contains cellulose and/or hemicellulose, into a carbon-based product, wherein the secondary raw material contains at least one pH regulator, said method comprising the step of bringing the secondary raw material into contact with a microorganism for a time period, at a starting temperature and an initial pH value, thereby producing an amount of lactic acid and/or of a different carbon-based product.

2. The method of claim 1, wherein the carbon-based product is a carboxylic acid, preferably lactic acid or a salt or ester thereof.

3. The method as per any one of the preceding claims of claim 1, wherein the secondary raw material is a papermaking residue containing cellulose and hemicellulose.

4. The method of claim 1, wherein the papermaking residue that contains cellulose and hemicellulose is deinking sludge.

5. The method of claim 1, wherein the papermaking residue that contains cellulose and hemicellulose is fiber waste, fiber sludge, filler sludge and coating sludge from mechanical separation.

6. The method of claim 1, wherein, other than the pH regulator already present in the secondary raw material, no additional pH regulator is added or only an amount of pH regulator is added to said method that contains fewer moles than the lactic acid produced.

7. The method of claim 1, wherein the pH regulator is CaCO₃.

8. The method of claim 1, wherein, during the fermentative conversion process, no enzymes are added to the method that degrade cellulose and/or hemicellulose.

9. The method of claim 1, wherein the secondary raw material containing cellulose and/or hemicellulose is not pretreated using enzymes that degrade cellulose and/or hemicellulose before the method.

10. The method of claim 1, wherein the microorganism belongs to the group of Thermoanaerobacterales.

11. The method of claim 1, wherein the microorganism belongs to the Caldicellulosiruptor or the Thermoanaerobacter genus.

12. The method of claim 1, wherein the microorganism is selected from the group consisting of DIB004C, deposited as DSM 25177, DIB041C, deposited as DSM 25771, DIB087C, deposited as DSM25772, DIB101C, deposited as DSM 25178, DIB103C, deposited as DSM 25773, DIB104C, deposited as DSM 25774, BluConL60, deposited as DSM 33252 and DIB107C, deposited as DSM 25775.

13. The method of claim 1, wherein the microorganism is selected from the group consisting of DIB004G, deposited as DSM 25179, DIB101G, deposited as DSM 25180, DIB101X, deposited as DSM 25181, DIB097X, deposited as DSM 25308, DIB087G, deposited as DSM 25777, DIB103X, deposited as DSM 25776, DIB104X, deposited as DSM 25778 and DIB107X, deposited as DSM 25779.

14. The method of claim 1, wherein the microorganism and an additional microorganism in the form of a coculture are brought into contact with the secondary raw material.

15. The method of claim 14, wherein the additional microorganism is also a microorganism that is mentioned in claims 10 to 13.

16. The method of claim 1, wherein the time period is 10 h to 300 h, preferably 50 h to 200 h, 70 h to 120 h, the starting temperature lies in the range of between 55° C. and 80° C., preferably between 65° C. and 72° C. and the initial pH value lies between 5 and 9, preferably between 6 and 8.

17. The method of claim 1, wherein the starting temperature is between 65° C. and 80° C., the time period is 120 hours or longer and the initial pH value is between 6 and 8.

18. The method of claim 1, wherein the carbon-based product is an alcohol, preferably ethanol.
